(19) United States
(12) Patent Application Publication
(10) Pub. No.: US 2024/0141281 A1
(43) Pub. Date: May 2, 2024

(54) INFORMATION PROCESSING DEVICE, DETACHMENT ASSISTANCE SYSTEM, LEARNING DEVICE, NON-TRANSITORY STORAGE MEDIUM, INFORMATION PROCESSING METHOD, AND CELL DETACHMENT METHOD

(71) Applicant: CANON KABUSHIKI KAISHA, Tokyo (JP)

(72) Inventors: KENJIRO TAKEMURA, Tokyo (JP); YUTA KURASHINA, Tokyo (JP); CHIKAHIRO IMASHIRO, Tokyo (JP); KAZUNORI NOGUCHI, Ibaraki (JP); KATSUHISA YAMAZAKI, Shizuoka (JP); MASASHI HIROSE, Tokyo (JP); KEIICHIRO TSUBAKI, Tokyo (JP); SUGURU WATANABE, Kanagawa (JP); AKIRA SUGIYAMA, Kanagawa (JP); KENICHI KAKU, Shizuoka (JP); TAKAAKI FURUI, Tokyo (JP); TATSUO FURUTA, Tokyo (JP); HITOMI TOKUTAKE, Kanagawa (JP); RYUICHI OTSU, Tokyo (JP); YUKARI NAKASHOJI, Saitama (JP)

(21) Appl. No.: 18/492,493

(22) Filed: Oct. 23, 2023

(30) Foreign Application Priority Data
Oct. 26, 2022 (JP) 2022-171672

(51) Int. Cl. C12M 1/36; C12M 1/34

(52) U.S. Cl. CPC C12M 41/48; C12M 41/46; C12N 5/0602

(57) ABSTRACT

The information processing device includes: a cultivation information acquisition unit configured to acquire cultivation information of a cell; and a recommended condition generation unit configured to generate a recommended condition for detaching the cell from a to-be-processed vessel in which the cell is cultured, the cultivation information including information about the to-be-processed vessel, the recommended condition being a condition for detaching the cell by applying vibration to the to-be-processed vessel, the recommended condition generation unit being configured to generate the recommended condition based on the cultivation information, with use of a learned model, the learned model being learned with use of information about a cultivation condition and information about a detachment condition, the information about the cultivation condition including information about a culture vessel, the information about the detachment condition including information about a condition for applying vibration to the culture vessel.

### BACKGROUND OF THE INVENTION

### Field of the Invention

The present invention relates to an information processing device, a detachment assistance system, a learning device, a non-transitory storage medium, an information processing method, and a cell detachment method.

### Description of the Related Art

Cell culture for culturing a cell to be used in regenerative medicine and a cell to be used in manufacture of a bio-pharmaceutical or the like is actively practiced. A cultured cell is provided for each use after being collected from a culture vessel.

In a case of an adherent cell, the cell is required to be detached from a culture vessel in order to collect the cell from the culture vessel. Methods of detaching a cell from a culture vessel include one in which the cell is detached by applying vibration to the culture vessel.

A condition under which vibration is applied to detach the cell has a possibility of affecting subsequent uses of the cell. To give a specific example, when cells can be detached at a high detachment rate, cultured cells can be efficiently used and cost and labor for preparing a cell for a specific use can be reduced. In a case of providing a collected cell for a use that utilizes cell activity, and in a case of providing a collected cell for subsequent culture, it is preferred to detach the cell under a condition that keeps the cell activity as high as possible, in particular, a condition that can raise a cell survival rate.

It is accordingly preferred to detach a cell by applying vibration under a condition well suited to a use. However, effects of the same detachment condition on cells cultured under different cultivation conditions vary. A detachment condition suited to each cultivation condition is accordingly required to be selected. A problem is that it requires considerable time and labor to search for and determine a cell detachment condition each time the cultivation condition changes.

In recent years, with advancement of information processing technology, attempts have been made to utilize information processing technology in cell culture technology for effective progress of work.

In International Publication No. WO2018/142702, there is disclosed a culturing assistance device for assessing a culture state of cells. The culture assistance device as described in International Publication No. WO2018/142702 includes a learning unit which learns a relationship between a photographic image of cells being cultured that is acquired at a prescribed time interval and an event pertaining to culturing of the cells.

In International Publication No. WO2020/039683, there is disclosed a cell culture support device for supporting cell culture from an initial culture step to a production culture step, which uses equipment greater in scale than equipment used for the initial culture step. The cell culture support device as described in International Publication No. WO2020/039683 is used for a purpose of shortening a period from the initial culture step to the production culture step.

None of the devices as described in International Publication No. WO2018/142702 and International Publication No. WO2020/039683 is suitable for use as a way to determine a condition under which a cultured cell is detached.

### SUMMARY OF THE INVENTION

It is accordingly an object of the present invention to provide an information processing device suitable for determination of an effective condition under which a cultured cell is detached by applying vibration.

Another object of the present invention is to provide a detachment assistance system, an information processing method, and a cell detachment method that use the above-mentioned information processing device.

Still another object of the present invention is to provide a learning device and a non-transitory storage medium having stored thereon an information processing program that are used in the above-mentioned information processing device.

The above-mentioned objects are achieved by the present invention described below.

That is, according to one aspect of the present invention, there is provided an information processing device including: a cultivation information acquisition unit configured to acquire cultivation information of a cell; and a recommended condition generation unit configured to generate a recommended condition for detaching the cell from a to-be-processed vessel in which the cell is cultured, the cultivation information including information about the to-be-processed vessel, the recommended condition being a condition for detaching the cell by applying vibration to the to-be-processed vessel, the recommended condition generation unit being configured to generate the recommended condition based on the cultivation information, with use of a learned model, the learned model being learned with use of information about a cultivation condition and information about a detachment condition, the information about the cultivation condition including information about a culture vessel, the information about the detachment condition including information about a condition for applying vibration to the culture vessel.

Further, according to one aspect of the present invention, there is provided a detachment assistance system including: a measurement device configured to execute measurement for acquisition of the cultivation information; and the above-mentioned information processing device, wherein the measurement device includes a vessel measurement device configured to execute measurement for acquisition of the information about the to-be-processed vessel.

Further, according to one aspect of the present invention, there is provided a learning device configured to: use, as input, a cultivation condition of a cell cultured in a culture vessel; use, as output, a detachment condition for detaching the cell from the culture vessel by applying vibration; and execute reinforcement learning of a machine learning model, with improvement of a detachment result as reward, the detachment result being observed when the cell cultured under the cultivation condition is detached under the detachment condition, wherein information about the cultivation condition includes information about the culture vessel, and wherein the improvement of the detachment result includes at least one of an increase in detachment rate and a rise in survival rate of the cell.

Further, according to one aspect of the present invention, there is provided a non-transitory storage medium having stored thereon an information processing program for causing a computer to: acquire cultivation information of a cell; and generate, based on the cultivation information, a recommended condition for detaching the cell from a to-be-processed vessel in which the cell is cultured, with use of a learned model, the cultivation information including information about the to-be-processed vessel, the recommended condition being a condition for detaching the cell by applying vibration to the to-be-processed vessel, the learned model being learned with use of information about a cultivation condition and information about a detachment condition, the information about the cultivation condition including information about a culture vessel, the information about the detachment condition including information about a condition for applying vibration to the culture vessel.

Further, according to one aspect of the present invention, there is provided an information processing method including: an information acquisition step of acquiring cultivation information of a cell; and a recommended condition generation step of generating a recommended condition for detaching the cell from a to-be-processed vessel in which the cell is cultured, the cultivation information including information about the to-be-processed vessel, the recommended condition being a condition for detaching the cell by applying vibration to the to-be-processed vessel, the recommended condition generation step including generating the recommended condition based on the cultivation information, with use of a learned model, the learned model being learned with use of information about a cultivation condition and information about a detachment condition, the information about the cultivation condition including information about a culture vessel, the information about the detachment condition including information about a condition for applying vibration to the culture vessel.

Further, according to one aspect of the present invention, there is provided a cell detachment method including: a recommended condition acquisition step of acquiring, with use of the above-mentioned information processing device, the recommended condition; a condition setting step of setting, based on the recommended condition, a processing condition for detachment a cell from the to-be-processed vessel; and a detachment step of detaching the cell from the to-be-processed vessel, based on the processing condition.

Further features of the present invention will become apparent from the following description of exemplary embodiments with reference to the attached drawings.

### BRIEF DESCRIPTION OF THE DRAWINGS

FIG. 1 is a function block diagram of an information processing device according to a first embodiment of the present invention.

FIG. 2 is a flow chart for illustrating an outline of processing to be executed by the information processing device according to the first embodiment of the present invention.

FIG. 3 is a block diagram for illustrating a hardware configuration of the information processing device according to the first embodiment of the present invention.

FIG. 4A, FIG. 4B, FIG. 4C, FIG. 4D, FIG. 4E, and FIG. 4F are schematic diagrams for illustrating examples of a culture vessel.

FIG. 5 is a schematic diagram for illustrating an operation example of the information processing device according to the first embodiment of the present invention.

FIG. 6 is a schematic diagram for illustrating an operation example of a display unit included in the information processing device according to the first embodiment of the present invention.

FIG. 7 is a function block diagram of an information processing device according to a second embodiment of the present invention.

FIG. 8 is a flow chart for illustrating an outline of processing to be executed by the information processing device according to the second embodiment of the present invention.

FIG. 9 is a function block diagram of an information processing device according to a third embodiment of the present invention.

FIG. 10 is a flow chart for illustrating an outline of processing to be executed by the information processing device according to the third embodiment of the present invention.

FIG. 11 is a function block diagram of a learning device according to a fourth embodiment of the present invention.

FIG. 12 is a function block diagram of a detachment assistance system according to a fifth embodiment of the present invention.

FIG. 13 is a flow chart for illustrating an outline of a cell detachment method that uses the detachment assistance system according to the fifth embodiment of the present invention.

### DESCRIPTION OF THE EMBODIMENTS

Exemplary embodiments of the present invention are now described with reference to the drawings. Like elements or corresponding elements are denoted by the same reference symbols in the drawings, and description thereof may be omitted or simplified.

### First Embodiment

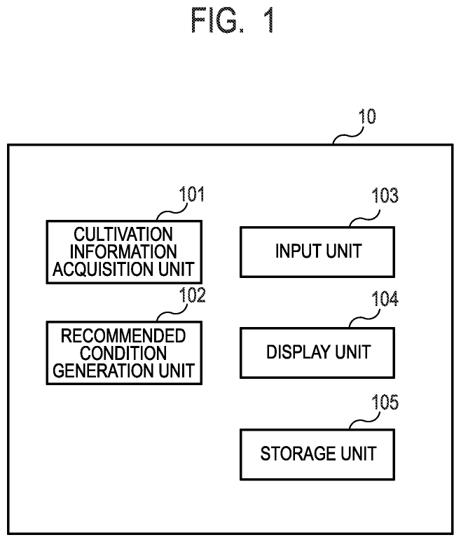

FIG. 1 is a block diagram of an information processing device 10 according to a first embodiment of the present invention.

As illustrated in FIG. 1, the information processing device 10 according to the present embodiment includes a cultivation information acquisition unit 101, a recommended condition generation unit 102, an input unit 103, a display unit 104, and a storage unit 105.

The information processing device 10 according to the present embodiment is used to generate, based on cultivation information of a cell, a recommended condition usable to determine a condition effective for detachment of the cell from a culture vessel. The information processing device 10 according to the present embodiment may include a display control unit (not shown).

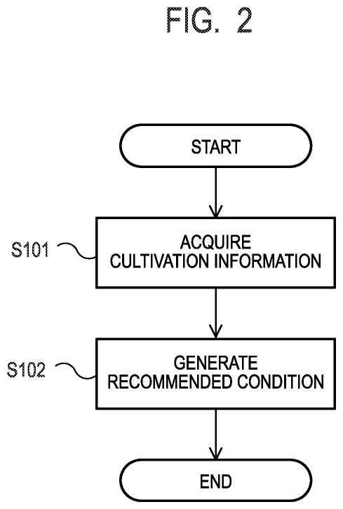

FIG. 2 is a flow chart for illustrating an outline of processing to be executed by the information processing device 10.

In Step S101, which is a cultivation information acquisition step, the cultivation information acquisition unit 101 acquires cultivation information of a cell based on input from the input unit 103. The cultivation information acquired by the cultivation information acquisition unit 101 includes information about a to-be-processed vessel from which the cell is to be detached. The information acquired by the cultivation information acquisition unit 101 is transmitted to the recommended condition generation unit 102. Prior to, or at the same time as, the transmission to the recommended condition generation unit 102, the acquired information may be stored in the storage unit 105. The cell to be detached here can be any of a single cell, an aggregate in which a plurality of cells clump together, and cells cultured in a shape of a sheet (a cell sheet) (the same applies to the following).

In Step S102, which is a recommended condition generation step, the recommended condition generation unit 102 generates, based on the cultivation information acquired by the cultivation information acquisition unit 101, a recommended condition for detaching the cell from the to-be-processed vessel in which the cell has been cultured. In the present embodiment, the recommended condition generation unit 102 uses a learned model stored in the storage unit 105 to generate the recommended condition. The learned model here is learned with use of information about cultivation conditions and information about detachment conditions, and the information about detachment conditions used in the learning by the learned model includes information about conditions under which vibration is applied to the culture vessel.

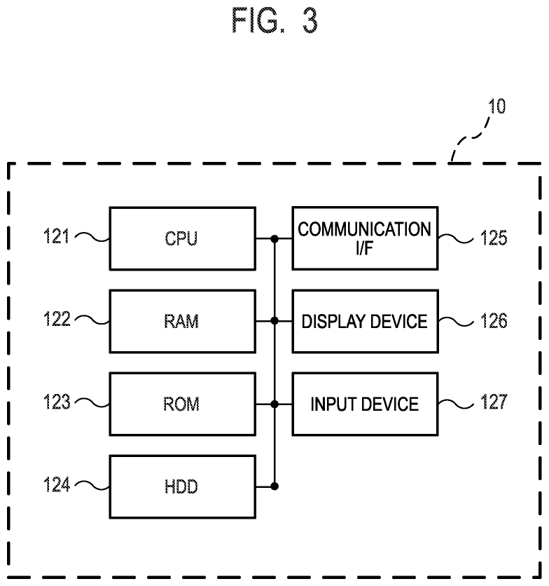

FIG. 3 is a block diagram for illustrating a hardware configuration of the information processing device 10 according to the present embodiment. The information processing device 10 includes a Central Processing Unit (CPU) 121, a Random Access Memory (RAM) 122, a Read Only Memory (ROM) 123, a hard disk drive (HDD) 124, a communication OF 125, a display device 126, and an input device 127. These units are interconnected via a bus or the like.

The CPU 121 is a processor that reads programs stored in the ROM 123 and the HDD 124 into the RAM 122 and executes the programs, and performs calculation processing, control of each unit of the information processing device 10, and the like. The processing performed by the CPU 121 may include acquisition of information of cultivation conditions, generation of a recommended condition, and the like.

The RAM 122 is a volatile storage medium and functions as a work memory when the CPU 121 executes a program. The ROM 123 is a non-volatile storage medium, and stores firmware and the like necessary for the operation of the information processing device 10. The HDD 124 is a non-volatile storage medium, and stores information acquired by the cultivation information acquisition unit 101, a learned model used by the recommended condition generation unit 102, programs used for acquisition of cultivation information or generation of a recommended condition, and the like.

The communication OF 125 is a communication device based on a standard such as Wi-Fi (registered trademark), Ethernet (registered trademark), or Bluetooth (registered trademark). The communication OF 125 is used for communication with a measurement device and a detachment device which will be described below, and another computer, or the like.

The input device 127 is a device for inputting information to the information processing device 10, and is typically a user interface for a user to operate the information processing device 10. Examples of the input device 127 include a keyboard, buttons, a mouse, and a touch panel.

The display device 126 is a device in which the information processing device 10 outputs information to the outside, and is typically a user interface for presenting information to the user. Examples of the display device 126 include a display and a speaker.

Note that the configuration of the information processing device 10 described above is merely an example, and can be changed as appropriate. Examples of processors that can be mounted on the information processing device 10 include a Graphics Processing Unit (GPU), an Application Specific Integrated Circuit (ASIC), and a Field Programmed Gate Array (FPGA) in addition to the CPU 121. A plurality of processors may be provided, or a plurality of processors may perform processing in a distributed manner. The function of storing information such as image data in the HDD 124 may be provided not in the information processing device 10 but in another data server. The HDD 124 may be a storage medium such as an optical disk, a magneto-optical disk, or a solid-state drive (SSD).

The CPU 121 executes a program to perform predetermined calculation processing. The CPU 121 controls each unit in the information processing device 10 by executing a program. Through these processes, the CPU 121 realizes the functions of the cultivation information acquisition unit 101 and the recommended condition generation unit 102.

The hardware configuration illustrated in FIG. 3 is an example, and devices other than the illustrated devices may be added, or part of the illustrated devices may be omitted as long as the functions of the information processing device 10 according to the present embodiment are realized. In addition, part of the devices may be substituted with another device having the same function. Further, part of the functions may be provided by another device via a network, and the functions for implementing the embodiments may be shared and implemented by a plurality of devices. For example, the HDD 124 may be substituted with a solid state drive (SSD) using a semiconductor element such as a flash memory.

An examination by the inventors of the present invention has revealed that, in determination of a condition for detaching a cell by applying vibration to a to-be-processed vessel, it is important to take information about the to-be-processed vessel in which the cell has been cultured into consideration. Accordingly, in the present embodiment, the information about cultivation condition used in the learning by the learned model includes information about the culture vessel.

The recommended condition generation unit 102 may automatically generate the recommended condition based on the information acquired by the cultivation information acquisition unit 101, or may generate the recommended condition based on input from the input unit 103.

The recommended condition generated by the recommended condition generation unit 102 may be stored in the storage unit 105.

The display unit 104 can display the information acquired by the cultivation information acquisition unit 101 and the recommended condition generated by the recommended condition generation unit 102. In a case in which a user of the information processing device 10 is not required to check the information acquired by the cultivation information acquisition unit 101 and the recommended condition generated by the recommended condition generation unit 102, the information processing device 10 may omit the display unit 104.

The information about the culture vessel used for the learning by the learned model may include at least one type of information selected from the group consisting of the following four types of information. That is, the four types of information are information about a type of the culture vessel, information about a shape of the culture vessel, information about a chemical property of the culture vessel, and information about a physical property of the culture vessel.

The inventors of the present invention have found out that culture vessels are commercially available from a plurality of companies, and that culture vessels similar to each other in form sometimes differ from each other in cell detachment property when the culture vessels are different in terms of manufacturing company, sales company, or part number. A reason for the varied detachment property from one culture vessel to another culture vessel is considered to be a difference in at least one of shapes, chemical properties, and physical properties of the culture vessels regardless of the similarity. For example, in a case of dishes purposed for culture of a hydrophilic cell, culture vessels similar to each other may differ in thickness of a bottom surface portion or a side surface portion, presence or absence of a convex portion in the side surface portion, or a shape, a material, or the like of the convex portion. According to the examination of the inventors of the present invention, a frequency of the vibration is preferred to be higher when the bottom surface portion increases in density, and a higher vibration intensity tends to be preferred when the side surface portion is thicker.

From the above-mentioned examination, the information about the type of the culture vessel may include at least one piece of information selected from the group consisting of information about a manufacturing company of the culture vessel, information about a sales company of the culture vessel, and information about a part number of the culture vessel. The information about the type of the culture vessel is not information directly related to characteristics of the culture vessel. However, in some cases, the culture vessel itself or features of the culture vessel such as a material and a shape can be identified based on the information about the type of the culture vessel. An effective condition for detachment can accordingly be determined based on the culture vessel or features of the culture vessel identified from the information about the type of the culture vessel.

Further, the information about the shape of the culture vessel may include at least one type of information selected from the group consisting of the following four types of information. The four types of information are information about a form of the culture vessel, information about a thickness of the culture vessel, information about a curvature of the bottom surface portion of the culture vessel, and information about a flatness and smoothness of an inner surface of the culture vessel.

Forms of the culture vessel include typical shapes of a flask, a dish, a well, and the like, and a cell can effectively be detached by applying vibration under a condition suitable for one of those shapes. Culture vessel types may include a dish type, and the information about the shape of the culture vessel may include information about presence or absence of a convex portion provided in a bottom surface edge portion of the dish. When there is a convex portion provided in the bottom surface edge portion of the dish, the information about the shape of the culture vessel may include information about a shape of the convex portion.

The thickness of the culture vessel and the curvature of the bottom surface portion affect transmission of the vibration in particular, and the flatness and smoothness of the inner surface of the culture vessel affect an adherence property of the cell. Accordingly, the cell can effectively be detached by applying vibration under a condition that takes those into consideration.

In addition, the information about the chemical property of the culture vessel may include at least one piece of information selected from the group consisting of information about a material forming the culture vessel, and information about chemical modification of the inner surface of the culture vessel.

Examples of the material forming the culture vessel include glass, resin, and stainless steel. Of those, polystyrene vessels are widely used. One polystyrene vessel and another polystyrene vessel may differ in characteristics such as density and Young's modulus. Those differences in material forming the culture vessel affect transmission of the vibration and the adherence property of the cell. For chemical modification of the inner surface of the culture vessel, fibronectin, collagen, lysine, laminin, and the like are commonly used. Thermo-responsive chemical modification and photo-responsive chemical modification are used in some cases. Those can affect the adherence property of the cell. Accordingly, the cell can effectively be detached by applying vibration under a condition that takes those into consideration.

Further, the information about the physical property of the culture vessel may include at least one type of information selected from the group consisting of the following four types of information. The four types of information are information about hydrophilic property of the inner surface of the culture vessel, information about a contact angle of the inner surface of the culture vessel, information about a surface charge of the inner surface of the culture vessel, and information about a natural vibration frequency of the culture vessel.

The hydrophilic property of the inner surface of the culture vessel, the contact angle of the inner surface of the culture vessel, and the surface charge of the inner surface of the culture vessel each affect the adherence property of the cell in particular. The natural vibration frequency of the culture vessel affects transmission of the vibration to the culture vessel. Accordingly, the cell can effectively be detached by applying vibration under a condition that takes those into consideration.

The natural vibration frequency of the culture vessel can be a vibration frequency of a vibration that has been applied at a time when the vibration frequency of the culture vessel reaches a local maximum during application of a vibration in a certain range of vibration frequencies to the culture vessel. The vibration frequency of an applied vibration at which the vibration frequency of the culture vessel reaches the local maximum may be calculated by simulation. When the vibration frequency of the culture vessel has a plurality of local maximum values, a maximum value out of the plurality of local maximum values can be selected. A local maximum value of the vibration frequency of the culture vessel is measurable with a laser doppler or the like.

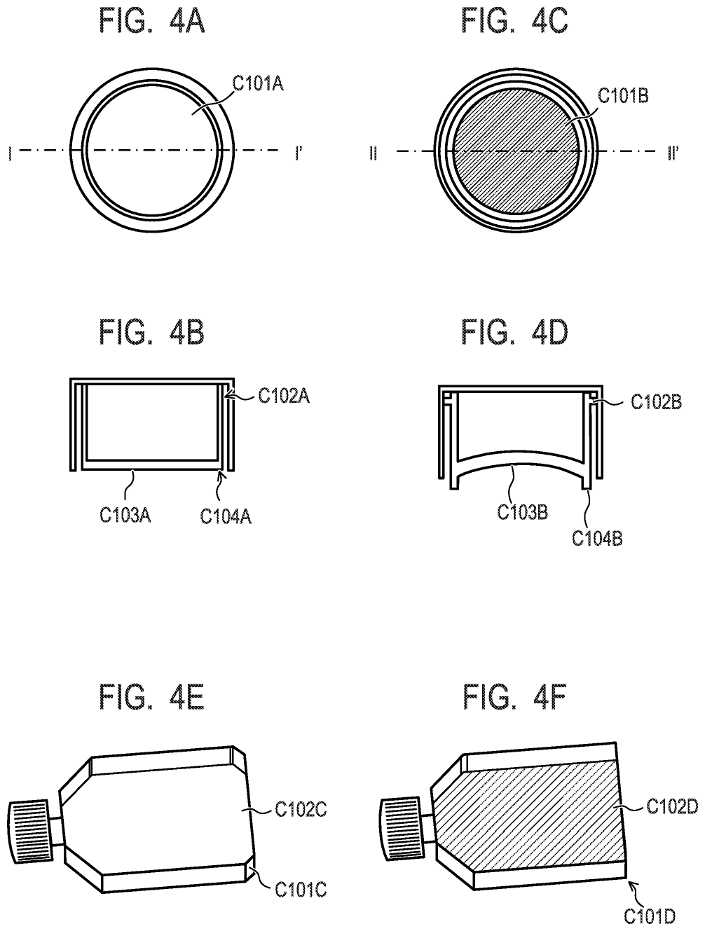

FIG. 4A to FIG. 4F are schematic diagrams for illustrating examples of the culture vessel. FIG. 4A is a top view of a culture vessel A. FIG. 4B is a schematic diagram of a vertical sectional view taken along the line of FIG. 4A. FIG. 4C is a top view of a culture vessel B. FIG. 4D is a schematic diagram of a vertical sectional view taken along the line II-IF of FIG. 4C. FIG. 4E is a perspective view of a culture vessel C. FIG. 4F is a perspective view of a culture vessel D.

The culture vessel A and the culture vessel B differ from each other in chemical modification and water contact angles of bottom surfaces of the vessels, shapes of circumferential wall surfaces C102A and C102B, thicknesses and curvatures of bottom surfaces C103A and C103B, and shapes of bottom surface edge portions C104A and C104B. The culture vessel A has no convex portions in the wall surface C102A and the bottom surface edge portions C104A. The culture vessel B, on the other hand, has convex portions in the wall surface C102B and the bottom surface edge portion C104B. The culture vessel C and the culture vessel D differ from each other in chemical modification and water contact angles of bottom surfaces C102C and C102D, and shapes of corners C101C and C101D.

As illustrated in the examples of the culture vessels A to D described above, features that may affect the adherence property of the cell and transmission of the vibration vary from one culture vessel to another culture vessel, and the cell can effectively be detached by applying vibration suited to those features.

The culture vessel from which the information about the culture vessel is acquired is preferred to include a versatile culture vessel, and may include at least one selected from the group consisting of a flask, a dish, and a well.

In addition to the information about the culture vessel, the information about cultivation conditions may further include at least one type of information selected from the group consisting of the following five types of information. The five types of information are information about a cell, information about a cell semination density, information about a culture fluid, information about a cultivation period, and information about a culture environment. Here, the information about cells may include information about cell species. The information about the culture fluid may include a quantity of the culture fluid, a composition of the culture fluid, and a replacement condition of the culture fluid. The information about the culture environment may include temperature, humidity, illuminance, a composition of a gas supplied to the culture vessel, a rate at which the gas is supplied to the culture vessel, a stirring velocity of the culture fluid, and a condition for shaking the culture vessel. Those can affect the adherence property of the cell in particular. Accordingly, the cell can effectively be detached by applying vibration under a condition that takes those into consideration.

In the present embodiment, as a matter of course, the information about cultivation conditions used in the learning by the learned model inevitably includes information of the same category as a category of at least some of the information acquired by the cultivation information acquisition unit 101. That is, when the cultivation information acquired by the cultivation information acquisition unit 101 is only information about the thickness of the culture vessel, for example, the information about cultivation conditions used in the learning inevitably includes information about the thickness of the culture vessel. This enables the recommended condition generation unit 102 to generate the recommended condition based on the cultivation information acquired by the cultivation information acquisition unit 101, with the use of the learned model.

Meanwhile, the information about conditions under which vibration is applied to the culture vessel may include at least one type of information selected from the group consisting of the following three types of information. The three types of information are information about an intensity of the vibration to be applied to the culture vessel, information about a frequency of the vibration to be applied to the culture vessel, and information about a time for which the vibration is applied to the culture vessel. The cell can effectively be detached by adjusting the intensity and frequency of the vibration to be applied to the culture vessel and the time for which the vibration is applied to the culture vessel, so as to suit the cultivation condition of the cell. In particular, when the vibration applied to the culture vessel is vibration caused by application of an ultrasonic wave that is generated by an ultrasonic transducer, the information about the intensity of the vibration to be applied to the culture vessel may include information about a voltage to be applied to the ultrasonic transducer.

The information about the detachment condition may further include, in addition to the information about the conditions under which the vibration is applied, at least one piece of information selected from the group consisting of information about a detachment fluid and information about an acoustic matching material. The cell can effectively be detached by adjusting the type and quantity of the detachment fluid added to the culture vessel when vibration is applied, the type of the acoustic matching material used when vibration is applied, and the like, so as to suit the cultivation condition of the cell.

The information about the detachment condition is typically information obtained based on information about a detachment result. The information about the detachment result here may include at least one piece of information selected from the group consisting of information about a detachment rate of the cell, information about a survival rate of the detached cell, information about a growth rate of the detached cell, and information about functionality of the detached cell. The information about the detachment result here corresponds to demanded detachment performance which varies depending on the use of the detached cell. That is, when a cultured cell is to be collected at as high a ratio as possible, for example, the information about detachment conditions used in the learning by the learning model may be information acquired based on the information about the detachment rate of the cell.

The learned model used by the recommended condition generation unit 102 is a model obtained by performing training (learning) in advance on a machine learning model that follows a freely selected machine learning algorithm, such as deep learning. The machine learning model here refers to a learning model by a machine learning algorithm. It does not mean that the learned model, which learns in advance, stops learning further, and additional learning may be executed. The additional learning may be executed after the device is installed in the place of use of the device.

The learned model may be a regression expression obtained by regression analysis, or may be a neural network model obtained by deep learning or the like, and the data structure, the learning algorithm, and the like of the learned model are not limited. The learned model may refer to a single neural network when the learned model is constructed from a neural network, and an input layer, a hidden layer, and an output layer are regarded as a unit of one neural network. In the case described above, the learned model may also refer to a combination of a plurality of neural networks. The learned model may be constructed from a combination of a plurality of multiple regression expressions, or from one multiple regression expression.

The learning performed in advance on the machine learning model may be supervised learning with use of appropriate training data (learning data), or reinforcement learning executed so as to maximize reward. The training data here refers to learning data, and is constructed from pairs of input data and output data. The training data and the reward may be defined so as to generate a recommended condition that yields a desired detachment result.

The recommended condition generation unit 102 may use one learned model or a plurality of learned models. When the recommended condition generation unit 102 uses, for example, a plurality of learned models obtained with the use of machine learning models different from each other, a plurality of recommended conditions can be obtained and a selection of conditions can be presented to the user.

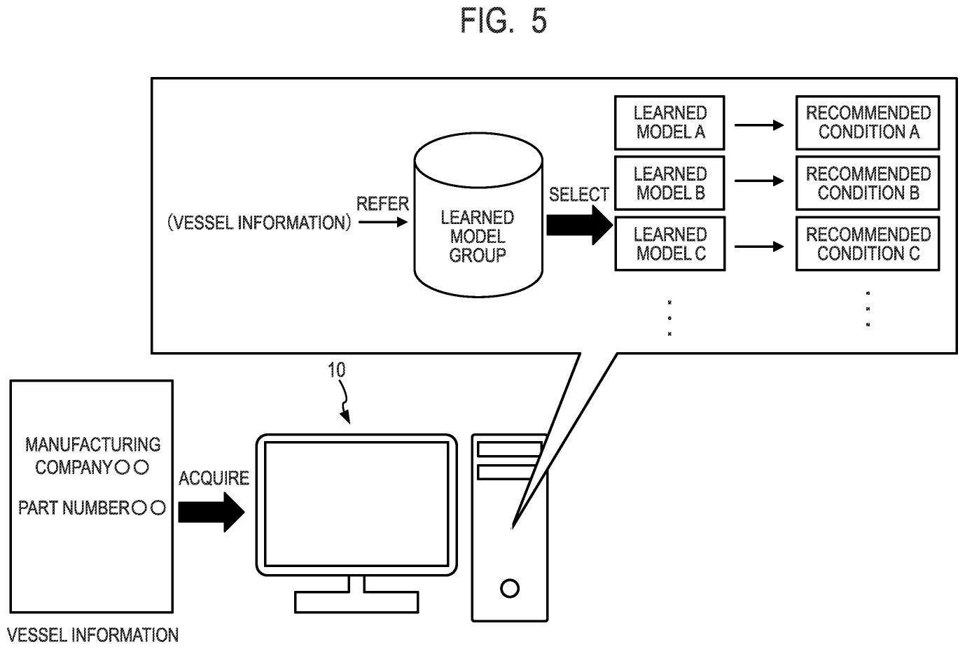

FIG. 5 is a schematic diagram for illustrating an operation example of the information processing device 10 according to the present embodiment.

As illustrated in FIG. 5, the cultivation information acquisition unit 101 included in the information processing device 10, for example, acquires vessel information about the to-be-processed vessel such as a manufacturing company and a part number as the cultivation information. The recommended condition generation unit 102 subsequently uses learned models A, B, and C, among others, selected from a learned model group stored in the storage unit 105 by input from the input unit 103, to generate recommended conditions A, B, and C, among others, from the learned models A, B, and C, respectively.

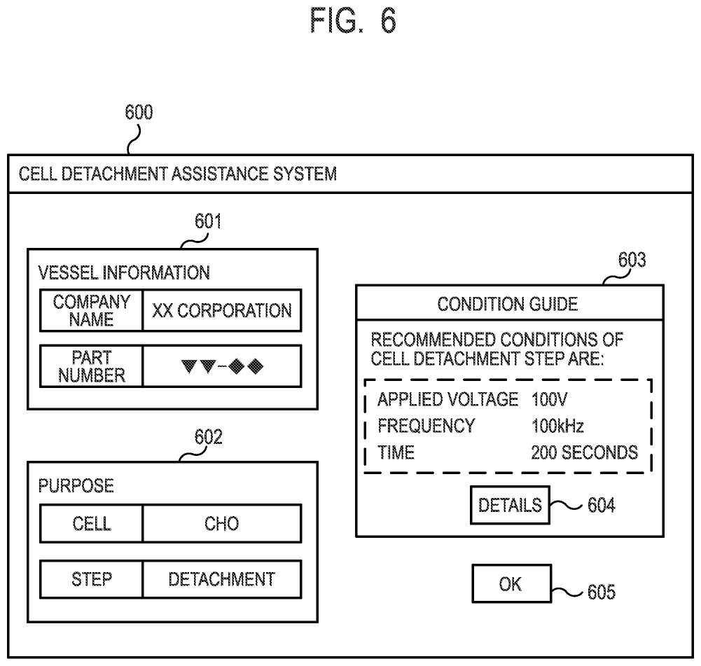

FIG. 6 is a schematic diagram for illustrating an operation example of the display unit 104 included in the information processing device 10 according to the present embodiment.

A display area of the display unit 104 displays a general display window 600 in which information about operation of the information processing device 10 according to the present embodiment is displayed in an integrated manner. The general display window 600 includes an acquired information display window 601, a purpose display window 602, a condition display window 603, and an approval button 605.

The acquired information display window 601 displays the vessel information and other pieces of cultivation information acquired by the cultivation information acquisition unit 101. The purpose display window 602 displays the type of a cell to be detached and a step. The condition display window 603 displays the recommended condition generated by the recommended condition generation unit 102. The condition display window 603 includes a detailed information transition button 604. When the user presses the detailed information transition button 604, a separate window which displays information about details of the recommended condition and details of the used learned model is displayed. When the user presses the approval button 605, the generated recommended condition is set as a condition for detaching the cell, and is stored in the storage unit 105.

Configurations and functions of the windows and the buttons illustrated in FIG. 6 are merely an example, and may be changed as required to suit functions demanded of the display unit 104. For example, when the cultivation information acquired by the cultivation information acquisition unit 101 includes information about the cell species, the type of the cell displayed in the purpose display window may be displayed in the acquired information display window 601. To give another example, when the information processing device 10 is an information processing device used in a detachment assistance system described later, the press of the approval button 605 may cause the detachment device to operate under the recommended condition.

The device described in the embodiment described above may be configured as in the following second embodiment and third embodiment.

### Second Embodiment

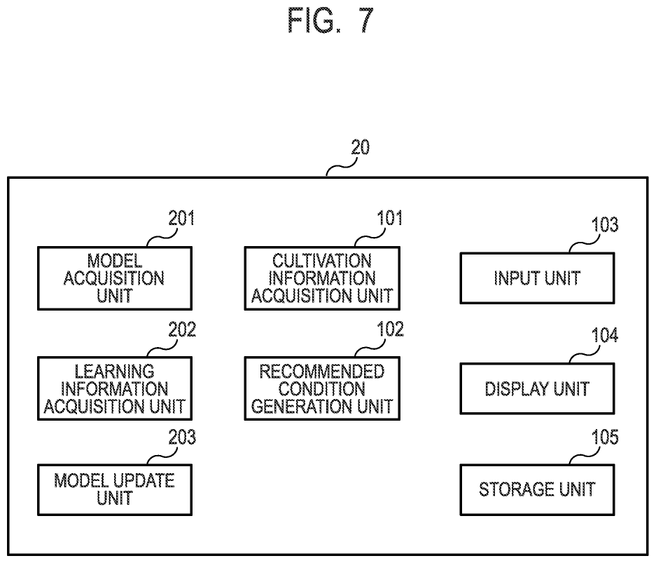

FIG. 7 is a block diagram of an information processing device 20 according to a second embodiment of the present invention. The information processing device 20 according to the present embodiment includes, in addition to the same components as the components included in the information processing device 10 according to the first embodiment, a model acquisition unit 201, a learning information acquisition unit 202, and a model update unit 203.

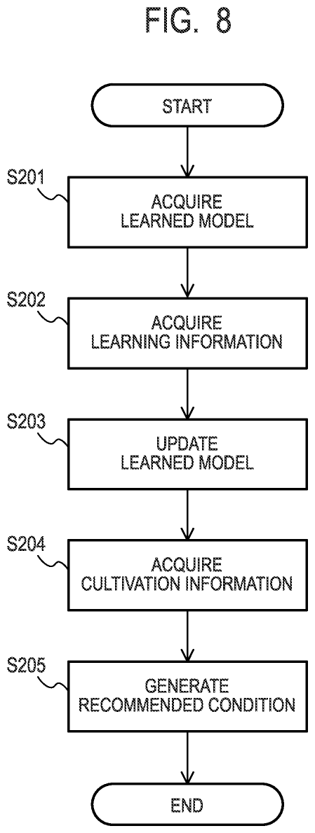

FIG. 8 is a flow chart for illustrating an outline of processing to be executed by the information processing device 20 according to the present embodiment.

In Step S201, which is a model acquisition step, the model acquisition unit 201 acquires a learned model from an external device via a network or the like, based on input of the input unit 103. The learned model acquired by the model acquisition unit 201 may be stored in the storage unit 105.

In Step S202, which is a learning information acquisition step, the learning information acquisition unit 202 acquires information to be used in additional learning of the learned model acquired by the model acquisition unit 201, based on input of the input unit 103. The learning information acquisition unit 202 may acquire the information for the additional learning from an external storage device via a network or the like, or may acquire the information from among pieces of information stored in the storage unit 105. For example, the learning information acquisition unit 202 may acquire, as the information for the additional learning, from the storage unit 105, the cultivation information stored in the storage unit 105 after being acquired by the cultivation information acquisition unit 101.

In the subsequent Step S203, which is a model update step, the model update unit 203 causes the learned model acquired by the model acquisition unit 201 to learn with use of the information for the additional learning acquired by the learning information acquisition unit 202, to thereby update the learned model. The learned model updated by the model update unit 203 may be stored in the storage unit 105.

In Step S204, which is a cultivation information acquisition step, the cultivation information acquisition unit 101 acquires the cultivation information of the cell based on input from the input unit 103.

In the subsequent Step S205, which is a recommended condition generation step, the recommended condition generation unit 102 generates, with use of the learned model updated by the model update unit 203, a recommended condition for detaching the cell from the to-be-processed vessel, based on the cultivation information acquired by the cultivation information acquisition unit 101.

A flow from Step S204 to Step S205 is the same as a flow from Step S101 to Step S102 in the first embodiment.

According to the present embodiment, the information processing device 20 can generate a recommended condition with the use of the learned model that has been updated with the latest information.

Step S201 and Step S202 may switch places in the order of execution. Step S204 may switch places with any of Step S201 to Step S203 in the order of execution.

### Third Embodiment

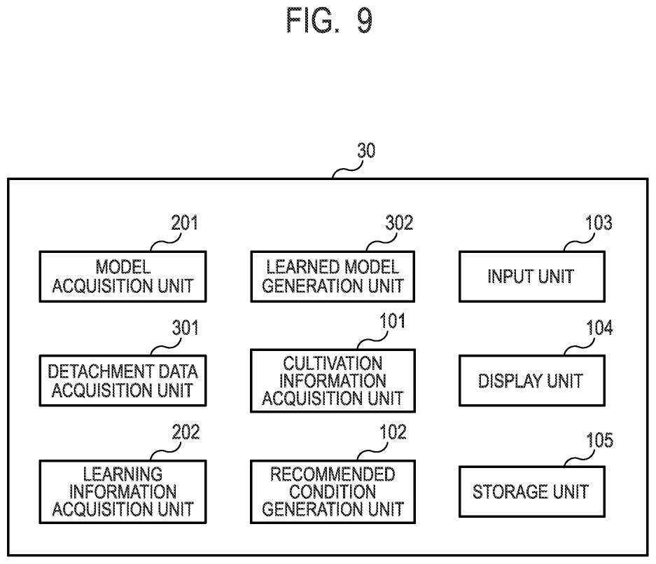

FIG. 9 is a block diagram of an information processing device 30 according to a third embodiment of the present invention. The information processing device 30 according to the present embodiment includes, in addition to the same components as the components included in the information processing device 10 according to the first embodiment, the model acquisition unit 201, a detachment data acquisition unit 301, the learning information acquisition unit 202, and a learned model generation unit 302.

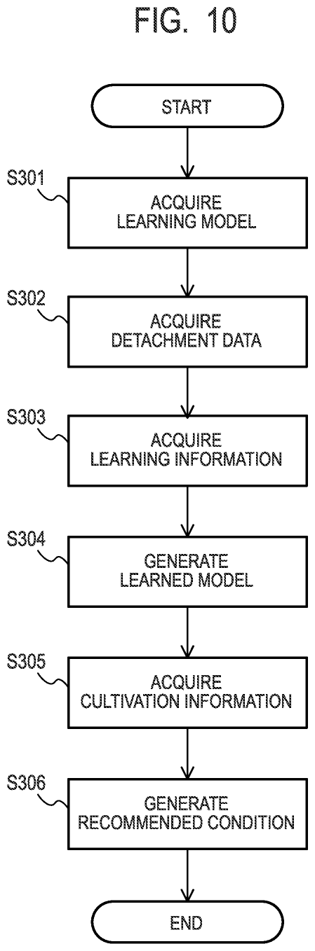

FIG. 10 is a flow chart for illustrating an outline of processing to be executed by the information processing device 30 according to the present embodiment.

In Step S301, which is a model acquisition step, the model acquisition unit 201 acquires a machine learning model from an external device via a network or the like, based on input of the input unit 103. The machine learning model acquired by the model acquisition unit 201 may be stored in the storage unit 105.

In Step S302, which is a detachment data acquisition step, the detachment data acquisition unit 301 acquires detachment data based on input from the input unit 103. The detachment data acquired by the detachment data acquisition unit 301 includes a combination of information about a cultivation condition, information about a detachment condition used to detach a cell that has been cultivated under that cultivation condition from the culture vessel, and information about a detachment result of detachment executed under the used detachment condition.

The information about the cultivation condition and the information about the detachment condition that are included in the detachment data are the same as the pieces of information described in the first embodiment as information usable for learning of the machine learning model. The information about the detachment result may include at least one piece of information selected from the group consisting of information about the detachment rate of the cell, information about the survival rate of the detached cell, information about a growth rate of the detached cell, and information about functionality of the detached cell. The detachment data acquisition unit 301 may acquire the detachment data from an external storage device via a network or the like, or may acquire the detachment data from among pieces of information stored in the storage unit 105.

In the subsequent Step S303, which is a learning information acquisition step, the learning information acquisition unit 202 acquires, from the detachment data acquired by the detachment data acquisition unit 301, information to be used in learning by the machine learning model. In the present embodiment, the information acquired by the learning information acquisition unit 202 includes a combination of the information about cultivation conditions and the information about detachment conditions. The learning information acquisition unit 202 can acquire the combination of the information about cultivation conditions and information about detachment conditions by selection based on the information about the detachment result included in the detachment data.

In the next Step S304, which is a learned model generation step, the learned model generation unit 302 causes the machine learning model acquired by the model acquisition unit 201 to learn with use of the information acquired by the learning information acquisition unit 202, and thus generates a learned model. Specifically, the machine learning model is caused to learn with a cultivation condition as input and a detachment condition as output.

The learning of the machine learning model caused by the learned model generation unit 302 may be supervised learning or reinforcement learning. For example, combinations of information about a cultivation condition and information about a detachment condition that correspond to a desired detachment result may be selected by the learning information acquisition unit 202 to be used as training data in learning by the machine learning model. The machine learning model may also learn by reinforcement learning in which improvement of a detachment result is a reward, with use of every combination of information about a cultivation condition and information about a detachment condition that is included in the detachment data. Here, improvement of a detachment result may include at least one of an increase in detachment rate and a rise in cell survival rate.

In the subsequent Step S305, which is a cultivation information acquisition step, the cultivation information acquisition unit 101 acquires the cultivation information of the cell to be detached. In the subsequent Step S306, which is a recommended condition generation step, the recommended condition generation unit 102 generates, with use of the learned model generated by the learned model generation unit 302, a recommended condition based on the cultivation information acquired by the cultivation information acquisition unit 101.

A flow from Step S305 to Step S306 is the same as a flow from Step S101 to Step S102 in the first embodiment.

According to the present embodiment, the information processing device 30 can generate a recommended condition for detachment after preparing a learned model on its own.

Step S301 and Step S302 may switch places in the order of execution. Step S305 may switch places with any of Step S301 to Step S304 in the order of execution, but it is preferred to finish Step S301 to Step S304 first in order to start detachment as soon as a cell is ready to be detached.

### Fourth Embodiment

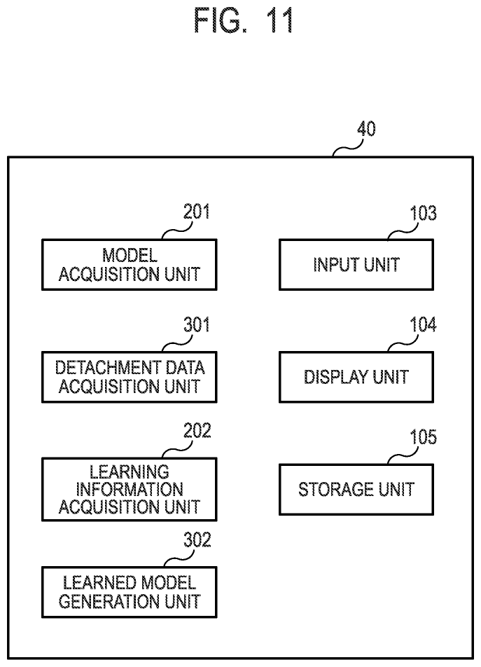

FIG. 11 is a block diagram of a learning device 40 according to a fourth embodiment of the present invention.

The learning device 40 according to the present embodiment includes, out of the components included in the information processing device 30 according to the third embodiment, the model acquisition unit 201, the detachment data acquisition unit 301, the learning information acquisition unit 202, the learned model generation unit 302, the input unit 103, the display unit 104, and the storage unit 105.

A flow executed by the learning device 40 according to the present embodiment is the same as a flow from Step S301 to Step S304 executed by the information processing device 30 according to the third embodiment.

According to the present embodiment, a learned model to be used by the information processing device 10 according to the first embodiment and the information processing device 20 according to the second embodiment can be provided.

### Fifth Embodiment

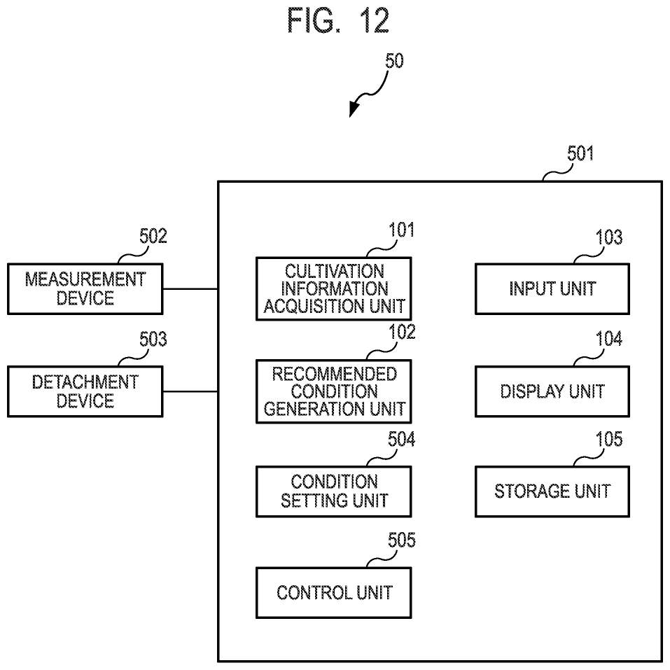

FIG. 12 is a function block diagram of a detachment assistance system 50 according to a fifth embodiment of the present invention.

The detachment assistance system 50 according to the present embodiment includes an information processing device 501, a measurement device 502, and a detachment device 503. The information processing device 501 further includes, in addition to the same components as the components included in the information processing device 10 according to the first embodiment, a condition setting unit 504 and a control unit 505.

The measurement device 502 is a component that executes measurement for acquisition of the cultivation information by the cultivation information acquisition unit 101. The measurement device 502 accordingly includes a vessel measurement device that executes measurement for acquisition of information about a to-be-processed vessel in which a cell has been cultured.

The detachment device 503 is a component for detachment, from the to-be-processed vessel, a cell that has been cultured in the to-be-processed vessel under a condition acquired by the information processing device 501. The detachment device 503 may be, for example, an ultrasonic transducer.

The condition setting unit 504 is a component for setting a processing condition under which the detachment device 503 detaches the cell from the to-be-processed vessel. The control unit 505 is a component for controlling the detachment device 503 based on the processing condition set by the condition setting unit 504.

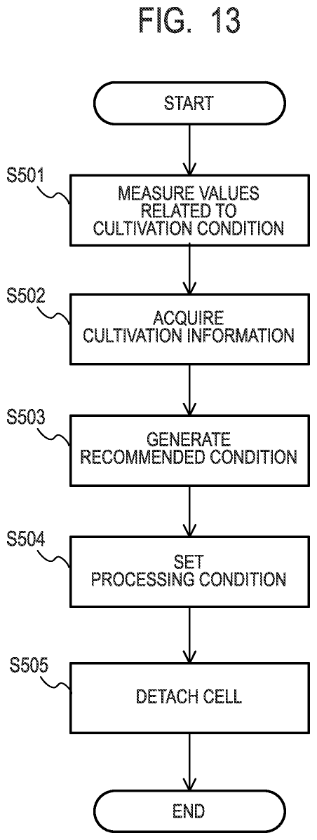

FIG. 13 is a flow chart for illustrating an outline of a cell detachment method that uses the detachment assistance system 50 according to the present embodiment.

First, in Step S501, which is a measurement step, the measurement device 502 measures values related to a cultivation condition of a cell to be detached. The measurement step of Step S501 includes a vessel measurement step in which the vessel measurement device included in the measurement device 502 executes measurement about the to-be-processed vessel.

In the subsequent Step S502, which is a cultivation information acquisition step, the cultivation information acquisition unit 101 acquires cultivation information. The cultivation information acquired by the cultivation information acquisition unit 101 includes the values measured by the measurement device 502.

In the following Step S503, which is a recommended condition generation step, the recommended condition generation unit 102 generates, based on the cultivation information acquired by the cultivation information acquisition unit 101, a recommended condition for detaching the cell from the to-be-processed vessel, with the use of a learned model.

A flow from Step S502 to Step S503 is the same as a flow from Step S101 to Step S102 in the first embodiment.

In the subsequent Step S504, which is a condition setting step, the condition setting unit 504 sets a processing condition for actually executing detachment, by referring to the recommended condition generated by the recommended condition generation unit 102.

The condition setting unit 504 may automatically perform required modification on the recommended condition, in accordance with criterion incorporated in advance, or the recommended condition may be changed based on the user's input from the input unit 103. For example, when the user determines the recommended condition generated with the use of the learned model to be unsuitable, a detachment condition may be set from an association relationship between the information about cultivation conditions and the information about detachment conditions, without using the learned model. The condition setting unit 504 may set the recommended condition as the processing condition without modification when the recommended condition does not require modification.

In the next Step S505, which is a detachment step, the detachment device 503 detaches the cell from the to-be-processed vessel under control of the control unit 505. Here, the control unit 505 controls the detachment device 503 based on the processing condition set by the condition setting unit 504.

Various modifications can be made to the detachment assistance system 50 described in the fifth embodiment.

For example, the information processing device 501 included in the detachment assistance system 50 may be replaced with one of the information processing devices 10 to 30 described in the first embodiment to the third embodiment, respectively, and a recommended condition acquisition step of acquiring a recommended condition with the use of one of the information processing devices 10 to 30 may be provided. Based on the acquired recommended condition, the user may manually set a processing condition for detaching the cell from the to-be-processed vessel in the condition setting step.

The detachment assistance system 50 may omit the condition setting unit 504, and the control unit 505 may control the detachment device 503 based on the recommended condition generated by the recommended condition generation unit 102, to thereby detach the cell from the to-be-processed vessel.

The detachment assistance system 50 may omit the control unit 505, and the user may manually perform required control on the detachment device 503 based on the processing condition set by the condition setting unit 504.

To give another example, the detachment assistance system 50 may include, in addition to the components illustrated in FIG. 12, a culture device for culturing a cell. Examples of the culture device include an incubator.

According to the present invention, it is possible to provide an information processing device suitable for determination of an effective condition under which a cultured cell is detached by applying vibration. Further, it is possible to provide a detachment assistance system, an information processing method, and a cell detachment method that use the above-mentioned information processing device. Still further, it is possible to provide a learning device and an information processing program that are used in the above-mentioned information processing device.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While preferable embodiments are explained above, the above-mentioned example embodiments merely describe specific examples in carrying out the present invention, and are not to be construed as limiting the technical scope of the present invention in any way. That is, the present invention can be implemented in various forms without departing from the technical idea or the main features of the present invention. For example, an embodiment which is produced by adding a part of constitutions of any of the embodiments to another embodiment, and an embodiment which is produced by replacing a part of constitutions of any of the embodiments with a part of another embodiment should be understood as to be embodiments capable of being applied with the present invention.

This application claims the benefit of Japanese Patent Application No. 2022-171672, filed Oct. 26, 2022, which is hereby incorporated by reference herein in its entirety.

What is claimed is:

1. An information processing device comprising:
a cultivation information acquisition unit configured to acquire cultivation information of a cell; and
a recommended condition generation unit configured to generate a recommended condition for detaching the cell from a to-be-processed vessel in which the cell is cultured,
the cultivation information including information about the to-be-processed vessel,
the recommended condition being a condition for detaching the cell by applying vibration to the to-be-processed vessel,
the recommended condition generation unit being configured to generate the recommended condition based on the cultivation information, with use of a learned model,
the learned model being learned with use of information about a cultivation condition and information about a detachment condition,
the information about the cultivation condition including information about a culture vessel,
the information about the detachment condition including information about a condition for applying vibration to the culture vessel.

2. The information processing device according to claim 1, wherein the information about the culture vessel includes at least one piece of information selected from the group consisting of information about a type of the culture vessel, information about a shape of the culture vessel, information about a chemical property of the culture vessel, and information about a physical property of the culture vessel.

3. The information processing device according to claim 2, wherein the information about the type of the culture vessel includes at least one piece of information selected from the group consisting of information about a manufacturing company of the culture vessel, information about a sales company of the culture vessel, and information about a part number of the culture vessel.

4. The information processing device according to claim 2, wherein the information about the shape of the culture vessel includes at least one piece of information selected from the group consisting of information about a form of the culture vessel, information about a thickness of the culture vessel, information about a curvature of a bottom surface portion of the culture vessel, and information about a flatness and smoothness of an inner surface of the culture vessel.

5. The information processing device according to claim 2, wherein the information about the chemical property of the culture vessel includes at least one piece of information selected from the group consisting of information about a material forming the culture vessel, and information about chemical modification of an inner surface of the culture vessel.

6. The information processing device according to claim 2, wherein the information about the physical property of the culture vessel includes at least one piece of information selected from the group consisting of information about hydrophilic property of an inner surface of the culture vessel, information about a contact angle of the inner surface of the culture vessel, information about a surface charge of the inner surface of the culture vessel, and information about a natural vibration frequency of the culture vessel.

7. The information processing device according to claim 1, wherein the culture vessel includes at least one selected from the group consisting of a flask, a dish, and a well.

8. The information processing device according to claim 2,
wherein the culture vessel includes a dish, and
wherein the information about the shape of the culture vessel includes information about presence or absence of a convex portion provided in a bottom surface edge portion of the dish.

9. The information processing device according to claim 8,
wherein the convex portion is provided in the bottom surface edge portion of the dish, and
wherein the information about the shape of the culture vessel includes information about a shape of the convex portion.

10. The information processing device according to claim 1, wherein the information about the condition for applying vibration to the culture vessel includes at least one piece of information selected from the group consisting of information about an intensity of the vibration to be applied to the culture vessel, information about a frequency of the vibration to be applied to the culture vessel, and information about a time for which the vibration is applied to the culture vessel.

11. The information processing device according to claim 10,
wherein the vibration applied to the culture vessel is vibration caused by application of an ultrasonic wave that is generated by an ultrasonic transducer, and
wherein the information about the intensity of the vibration to be applied to the culture vessel includes information about a voltage to be applied to the ultrasonic transducer.

12. The information processing device according to claim 1, wherein the information about the detachment condition further includes at least one piece of information selected from the group consisting of information about a detachment fluid and information about an acoustic matching material.

13. The information processing device according to claim 1,
wherein the information about the detachment condition is information obtained based on information about a detachment result, and
wherein the information about the detachment result includes at least one piece of information selected from the group consisting of information about a detachment rate of the cell, information about a survival rate of the detached cell, information about a growth rate of the detached cell, and information about functionality of the detached cell.

14. The information processing device according to claim 1, wherein the information about the cultivation condition further includes at least one piece of information selected from the group consisting of information about the cell, information about a cell semination density, information about a culture fluid, information about a cultivation period, and information about a culture environment.

15. The information processing device according to claim 14, wherein the information about the cell includes information about cell species.

16. The information processing device according to claim 1, further comprising a model update unit configured to update the learned model.

17. A detachment assistance system comprising:
a measurement device configured to execute measurement for acquisition of the cultivation information; and
the information processing device of claim 1,
wherein the measurement device includes a vessel measurement device configured to execute measurement for acquisition of the information about the to-be-processed vessel.

18. The detachment assistance system according to claim 17, further comprising a detachment device configured to detach a cell cultured in the to-be-processed vessel from the to-be-processed vessel.

19. The detachment assistance system according to claim 18, wherein the information processing device further includes a condition setting unit configured to set a processing condition under which the detachment device detaches the cell from the to-be-processed vessel.

20. The detachment assistance system according to claim 18, wherein the information processing device further includes a control unit configured to control the detachment device.

21. The detachment assistance system according to claim 18, wherein the detachment device includes an ultrasonic transducer.

22. A learning device configured to:
use, as input, a cultivation condition of a cell cultured in a culture vessel;
use, as output, a detachment condition for detaching the cell from the culture vessel by applying vibration; and
execute reinforcement learning of a machine learning model, with improvement of a detachment result as reward, the detachment result being observed when the cell cultured under the cultivation condition is detached under the detachment condition,
wherein information about the cultivation condition includes information about the culture vessel, and
wherein the improvement of the detachment result includes at least one of an increase in detachment rate and a rise in survival rate of the cell.

23. A non-transitory storage medium having stored thereon an information processing program for causing a computer to:
acquire cultivation information of a cell; and
generate, based on the cultivation information, a recommended condition for detaching the cell from a to-be-processed vessel in which the cell is cultured, with use of a learned model,
the cultivation information including information about the to-be-processed vessel,
the recommended condition being a condition for detaching the cell by applying vibration to the to-be-processed vessel,
the learned model being learned with use of information about a cultivation condition and information about a detachment condition,
the information about the cultivation condition including information about a culture vessel,
the information about the detachment condition including information about a condition for applying vibration to the culture vessel.

24. An information processing method comprising:
an information acquisition step of acquiring cultivation information of a cell; and
a recommended condition generation step of generating a recommended condition for detaching the cell from a to-be-processed vessel in which the cell is cultured,
the cultivation information including information about the to-be-processed vessel,
the recommended condition being a condition for detaching the cell by applying vibration to the to-be-processed vessel,
the recommended condition generation step including generating the recommended condition based on the cultivation information, with use of a learned model,
the learned model being learned with use of information about a cultivation condition and information about a detachment condition,
the information about the cultivation condition including information about a culture vessel,
the information about the detachment condition including information about a condition for applying vibration to the culture vessel.

25. A cell detachment method comprising:
a recommended condition acquisition step of acquiring, with use of the information processing device of claim 1, the recommended condition;
a condition setting step of setting, based on the recommended condition, a processing condition for detaching a cell from the to-be-processed vessel; and
a detachment step of detaching the cell from the to-be-processed vessel, based on the processing condition.

26. The cell detachment method according to claim 25, further comprising a measurement step of executing measurement for acquisition of the cultivation information,
wherein the measurement step includes executing measurement for acquisition of the information about the to-be-processed vessel.

27. The cell detachment method according to claim 25, wherein the detachment step includes detaching the cell with use of an ultrasonic transducer.
